(19) United States
(12) Patent Application Publication
(10) Pub. No.: US 2023/0057282 A1
(43) Pub. Date: Feb. 23, 2023

(54) COMPOSITIONS COMPRISING DESOXIMETASONE AND TAZAROTENE

(71) Applicant: TARO PHARMACEUTICAL INDUSTRIES LTD., Haifa Bay (IL)

(72) Inventors: Xiaopin JIN, Mississauga (CA); Bean NEZIR, Toronto (CA); Brenden HADJIKEZIAN, Vaughan (CA); Shen GAO, Bolton (CA); Ron SCHLINGER, Tel Aviv (IL); Avi AVRAMOFF, Karmeliya, Haifa (IL)

(21) Appl. No.: 17/758,783

(22) PCT Filed: Dec. 30, 2020
(86) PCT No.: PCT/US2020/067406

§ 371 Date: Jul. 13, 2022

Related U.S. Application Data
(60) Provisional application No. 62/972,891, filed on Feb. 11, 2020.

(51) Int. Cl. A61K 31/573; A61K 31/4436; A61K 47/59; A61K 47/34; A61K 9/00; A61K 47/44

(52) U.S. Cl. CPC A61K 31/573; A61K 31/4436; A61K 47/59; A61K 47/34; A61K 9/0014; A61K 47/44

(57) ABSTRACT

The present disclosure provides formulations comprising desoximetasone, tazarotene, a polymer comprising polyurethane, and a solvent. Also provided herein are methods of co-administering desoximetasone and tazarotene.

### FIELD OF THE INVENTION

The present disclosure provides formulations comprising desoximetasone, tazarotene, a polymer comprising polyurethane, and a solvent. Also provided herein are methods of co-administering desoximetasone and tazarotene.

### BACKGROUND

Desoximetasone is a corticosteroid that can be used for the treatment of skin conditions, such as rashes, redness, itching, irritation, inflammation, and the like. Corticosteroids are widely used to treat anti-inflammatory and many other medical conditions. Long term corticosteroid therapy may lead to suppression of the hypothalamic-pituitary-adrenal (HPA) axis. Typically, the HPA axis recovers after cessation of corticosteroids, but timing of recovery can be variable. Therefore, the main challenge in the development of any new treatment using corticosteroids is the development of effective treatment with low rates of HPA axis suppression and fast recovery

Tazarotene is a retinoid, typically topical, that can be used for the treatment of skin conditions such as psoriasis, acne, photodamage, and the like. The currently marketed formulation, TAZORAC® (Allergan, Irvine, Calif.), contains 0.05% or 0.1% tazarotene. A common side effect of tazarotene is dry, flaky skin. Thus, it is often recommended that tazarotene be applied in combination with a moisturizer or occlusive agent (see, e.g., TAZORAC FDA Label Information, February 2011; and “Tazarotene (Topical Route),” MayoClinic.org article dated Mar. 1, 2017, accessed on Sep. 16, 2019: www.mayoclinic.org/drugs-supplements/tazarotene-topical-route/proper-use/drg-20067364).

### SUMMARY OF THE INVENTION

In some embodiments, the present disclosure provides a topical formulation comprising: (a) about 0.01% wt/wt to about 0.25% wt/wt desoximetasone; (b) about 0.01% wt/wt to about 0.1% wt/wt tazarotene; (c) a polymer comprising polyurethane; and (d) a solvent.

In some embodiments, the polymer is a bis-urethane polyol polymer. In some embodiments, the polymer comprising polyurethane comprises a polyether. In some embodiments, the polymer comprising polyurethane comprises polypropylene glycol.

In some embodiments, the polymer comprises C₆-C₁₄ isocyanate and a polymer of propylene glycol. In some embodiments, the C₆-C₁₄ isocyanate is selected from trimethylhexanediisocyanate (TMHDI), saturated methylene diphenyldiisocyanate (SMDI), and hexamethylene diisocyante (HDI) trimer. In some embodiments, the C₆-C₁₄ isocyanate is saturated methylene diphenyldiisocyanate (SMDI).

In some embodiments, the polymer of propylene glycol comprises 6 to 20 units of propylene glycol. In some embodiments, the polymer of propylene glycol comprises 10 to 14 units of propylene glycol. In some embodiments, the polymer of propylene glycol comprises 12 units of propylene glycol.

In some embodiments, the C₆-C₁₄ isocyanate is saturated methylene diphenyldiisocyanate (SMDI) and the polymer of propylene glycol comprises 12 units of propylene glycol.

In some embodiments, the polymer is about 0.02% wt/wt to about 5% wt/wt. In some embodiments, the polymer is about 0.5% wt/wt to about 2% wt/wt. In some embodiments, the polymer is about 1% wt/wt to about 2% wt/wt.

In some embodiments, the solvent is selected from glycol, ethylene glycol, propylene glycol, isoprene glycol, butylene glycol, pentylene glycol, hexylene glycol, caprylyl glycol, and combinations thereof. In some embodiments, the solvent is hexylene glycol.

In some embodiments, the solvent is about 0.1% wt/wt to about 10% wt/wt, about 0.2% wt/wt to about 10% wt/wt. In some embodiments, the solvent is about 2% wt/wt to about 6% wt/wt.

In some embodiments, the desoximetasone is soluble in the solvent. In some embodiments, the tazarotene is soluble in the solvent. In some embodiments, the desoximetasone and the tazarotene are both soluble in the solvent.

In some embodiments, the formulation further comprises an ointment base, a surfactant, a rheology modifier, a penetration enhancer, or combinations thereof. In some embodiments, the ointment base is petrolatum.

In some embodiments, the ointment base is greater than 60% wt/wt of the formulation.

In some embodiments, the tazarotene is soluble in the ointment base.

In some embodiments, the surfactant is selected from propylene glycol stearate, glyceryl monohydroxystearate, isosteareth-2, trideceth-2, trideceth-3, hydroxystearic acid, PEG-2 stearate, sorbitan monostearate, glyceryl laurate, laureth-2, cocamide monoethanolamine, lauramide monoethanolamine, and combinations thereof. In some embodiments, the surfactant is propylene glycol stearate.

In some embodiments, the rheology modifier is selected from an acrylate crosspolymer; carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC), cyclomethicone, dimethicone, dicapryl maleate, caprylic/capric triglyceride, isopropyl myristate, octyl stearate, isostearyl linoleate, medium chain triglycerides (lanolin oil, coconut oil, cocoa butter, olive oil, avocado oil, aloe extracts, jojoba oil, castor oil), fatty acid, oleic acid, stearic acid, fatty alcohol, cetyl alcohol, hexadecyl alcohol, diisopropyl adipate, hydroxybenzoate esters, benzoic acid esters, isononyl iso-nonanoate, alkanes, mineral oil, silicone, dimethyl polysiloxane, ether, polyoxypropylene butyl ether, polyoxypropylene cetyl ether, and combinations thereof. In some embodiments, the rheology modifier is castor oil. In some embodiments, the rheology modifier is coconut oil.

In some embodiments, the penetration enhancer is selected from propylene glycol diesters of caprylic and capric acid, diethylene glycol monoethyl ether, or combinations thereof.

In some embodiments, the present disclosure provides a topical pharmaceutical formulation comprising: (a) about 0.03% wt/wt to about 0.15% wt/wt desoximetasone; (b) about 0.05% wt/wt to about 0.1% wt/wt tazarotene; (c) about 0.02% wt/wt to about 5% wt/wt polymer comprising C₆-C₁₄ isocyanate and polypropylene glycol; and (d) about 0.2% wt/wt to about 10% wt/wt organic, polyol solvent.

In some embodiments, the formulation further comprises (e) about 60% wt/wt to about 95% wt/wt ointment base, (f) about 2% wt/wt to about 10% wt/wt surfactant; and (g) about 2% wt/wt to about 15% wt/wt rheology modifier.

In some embodiments, the present disclosure provides a method of co-administering desoximetasone and tazarotene, the method comprising topically administering a formulation comprising desoximetasone; tazarotene; a polymer comprising polyurethane; and a solvent, wherein the desoximetasone skin permeation is inhibited at least 20% compared to a formulation not comprising the polymer.

In some embodiments, the skin permeation of the tazarotene is not substantially inhibited compared to a formulation not comprising the polymer.

In some embodiments, the present disclosure provides a method of co-administering desoximetasone and tazarotene, the method comprising topically administering a formulation comprising desoximetasone; tazarotene; a polymer comprising polyurethane; and a solvent, wherein the hypothalamic pituitary adrenal (HPA) axis suppression is reduced at least 10% compared to a formulation not comprising the polymer.

### BRIEF DESCRIPTION OF THE DRAWINGS

FIGS. 1A and 1B relate to the skin permeation study described in Example 2. FIG. 1A shows the amount of desoximetasone deposited into the skin (tape and skin data), or permeated into the receptor cell, from formulations containing different amounts of polymer PPG-12/SMDI. FIG. 1B shows tazarotene amounts measured in skin top layers (tapes) and deeper layers (skin) after washing the residual formulation from the skin surface from the same formulations.

FIG. 2 relates to the skin permeation study described in Example 3. FIG. 2A shows the amount of desoximetasone remaining in the skin top layers (tape), the skin deeper layers (Skin), or permeated through the skin, with ointment formulations containing 0.15% (Formulation 6) and 0.075% (Formulation 4) desoximetasone in the presence and absence of PPG-12/SMDI respectively. FIG. 2B shows the amount of tazarotene remaining in the skin top layers (tape), and the skin deeper layers (Skin), with ointment formulations containing 0.1% tazarotene in the presence and absence of PPG-12/SMDI.

### DETAILED DESCRIPTION OF THE INVENTION

The present disclosure relates to formulations comprising desoximetasone, tazarotene, a polymer comprising polyurethane, and a solvent.

As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability, depending on the situation. In some embodiments, one of skill in the art will understand the level of variability indicated by the term “about,” due to the context in which it is used herein. It should also be understood that use of the term “about” also includes the specifically recited value.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited elements or method steps. It is contemplated that any embodiment discussed in this specification can be implemented with respect to any composition (e.g., formulation) or method of the present disclosure. Furthermore, compositions (e.g., formulations) of the present disclosure can be used to achieve methods of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

The term “skin delivery” represents skin permeation and/or skin penetration. In vitro testing performed using human skin as a model membrane to study the listed formulations was used. In this test a full mass balance was pursued with quantification of the active performed on the skin surface (wash), in the top layers of the skin (Tape), epidermal layers of the skin (skin) and in the receptor cell as amount permeated through the skin. In some embodiments, the term “skin penetration” included the quantities of the active agent, within the skin layers without approaching the receptor cell. In some embodiments, the term “skin permeation” included the quantities of the active agent permeated through the skin.

In some embodiments, the present disclosure provides a topical formulation comprising: (a) about 0.03% wt/wt to about 0.15% wt/wt desoximetasone; (b) about 0.05% wt/wt to about 0.1% wt/wt tazarotene; (c) a polymer comprising polyurethane; and (d) a solvent.

In some embodiments, the disclosure provides that a corticosteroid other than desoximetasone can be used, i.e., formulations comprising a combination of a corticosteroid (that is not desoximetasone) and tazarotene are disclosed herein, as well as the use thereof, and the advantages of such formulations as described herein. In some embodiments, the present disclosure provides a topical formulation comprising: (a) about 0.03% wt/wt to about 0.15% wt/wt corticosteroid; (b) about 0.05% wt/wt to about 0.1% wt/wt tazarotene; (c) a polymer comprising polyurethane; and (d) a solvent.

Various corticosteroids are known in the art and can be used in the present invention. In some embodiments, the corticosteroid is a Class I, Class II, Class III, Class IV, Class V, Class VI, or Class VIII steroid. In some embodiments, the corticosteroid is a Class II corticosteroid. In some embodiments, the corticosteroid can include, but is not limited to, clobetasol propionate, halobetasol propionate, diflorasone diacetate, betamethasone dipropionate, fluocinonide, halcinonide, amcinonide, mometasone furoate, fluticasone propionate, triamcinolone acetonide, fluocinolone acetonide, hydrocortisone valerate, hydrocortisone butyrate, prednicarbate, hydrocortisone probutate, desonide, or combinations thereof.

The combination of desoximetasone and tazarotene was discovered to have advantageous effects on the treatment of skin conditions. However, methods of ameliorating the side effects of each active pharmaceutical ingredient (API) are generally incompatible. For example, to alleviate dry skin caused by tazarotene, a moisturizer (e.g., occlusive agent) may be desired, yet an occlusive agent may consequently lead to undesirable increase in the systemic absorption of desoximetasone.

In some embodiments, the present disclosure provides an improved desoximetasone and tazarotene topical pharmaceutical formulation that includes, but is not limited to, the following characteristics as compared to commercially available desoximetasone and/or tazarotene topical formulations: reduced skin permeation of desoximetasone; reduced hypothalamic-pituitary-adrenal (HPA) axis suppression; increased patient safety; and improved pharmacological or clinical efficacy.

In some embodiments, the formulation comprises a polymer comprising polyurethane. As used herein, the term “polymer” refers to a multimer derived from more than one species of monomer. Polymers described herein are not limited to how their monomeric units are arranged, e.g., in a linear chain including a single main chain, a branched chain with a single main chain and one or more polymeric chains, or grafted, star shaped, or any other architecture or arrangement. In some embodiments, the polymer is a block copolymer, e.g., two or more homopolymer subunits linked by covalent bonds. In some embodiments, the polymer is an alternating polymer, e.g., comprising regular alternating monomer units. In some embodiments, the polymer is a periodic polymer, e.g., wherein each repeating unit comprises two (or more) monomeric species. In some embodiments, the polymer is a statistical polymer. In some embodiments, the polymer is a stereoblock polymer. In some embodiments, the polymer is a gradient polymer.

In some embodiments, the polymer of the formulation comprises polyurethane. As used herein, polyurethane refers to a polymer comprising organic units joined by carbamate (also known as urethane) linkages. In some embodiments, the polymer is a bis-urethane polyol polymer. In some embodiments, bis-urethane comprises two isocyanate functional groups. In some embodiments, the polymer comprising polyurethane comprises an isocyanate and a polyol. In some embodiments, the isocyanate comprises two or more isocyanate functional groups. As used herein, isocyanates can refer to, e.g., diisocyanate, triisocyanate, or polyisocyanate. Exemplary isocyanates for polyurethane include, but are not limited to, aromatic diisocyanate, toluene diisocyanate (TDI), methylene diphenyl diisocyanate (MDI), trimethylhexanediisocyanate (TMHDI), saturated methylene diphenyldiisocyanate (SMDI), hexamethylene diisocyanate (HDI), isophorone diisocyanate (IPDI), and methyl isocyanate (MIC).

In some embodiments, the polyol of the polyurethane is a polyether polyol. Polyether polyols can be made, for example, from the reaction of epoxide with an active hydrogen-containing compound. In some embodiments, the polyol of the polyurethane is a polyester polyol. Polyester polyols can be made, for example, from the polycondensation of multifunctional carboxylic acids and polyhydroxyl compounds. Non-limiting examples of polyols for polyurethane include, but are not limited to, polybutyleneglycol, polypropylene glycol, polyethylene glycol, polycarbonate polyol, polycaprolactone polyol, polybutadiene polyol, and polysulfide polyol. In some embodiments, the polymer comprising polyurethane comprises polyether. In some embodiments, the polymer comprising polyurethane comprises polypropylene glycol.

In some embodiments, the polymer comprising polyurethane comprises isocyanate and a polyether polyol. In some embodiments, the isocyanate is aromatic diisocyanate, toluene diisocyanate (TDI), methylene diphenyl diisocyanate (MDI), trimethylhexanediisocyanate (TMHDI), saturated methylene diphenyldiisocyanate (SMDI), hexamethylene diisocyanate (HDI), isophorone diisocyanate (IPDI), or methyl isocyanate (MIC). In some embodiments, the isocyanate is a C₄-C₂₀ isocyanate. In some embodiments, the isocyanate is a C₆-C₁₄ isocyanate. In some embodiments, the isocyanate is a C₄, C₅, C₆, C₇, C₈, C₉, C₁₀, C₁₁, C₁₂, C₁₃, C₁₄, C₁₅, C₆, C₁₇, C₁₈, C₁₉, or C₂₀ isocyanate. In some embodiments, the polymer comprising polyurethane comprises C₆-C₁₄ isocyanate and a polymer of propylene glycol. In some embodiments, the C₆-C₁₄ isocyanate comprises trimethylhexanediisocyanate (TMHDI), saturated methylene diphenyldiisocyanate (SMDI), or hexamethylene diisocyante (HDI) trimer. In some embodiments, the C₆-C₁₄ isocyanate is saturated methylene diphenyldiisocyanate (SMDI).

In some embodiments, the polymer comprises C₆-C₁₄ isocyanate and a polymer of propylene glycol, wherein the polymer of propylene glycol comprises 2 to 60 units of propylene glycol. In some embodiments, the polymer of propylene glycol comprises 4 to 40 units of propylene glycol. In some embodiments, the polymer of propylene glycol comprises 6 to 20 units of propylene glycol. In some embodiments, the polymer of propylene glycol comprises 8 to 15 units of propylene glycol. In some embodiments, the polymer of propylene glycol comprises 10 to 14 units of propylene glycol. In some embodiments, the polymer of propylene glycol comprises 12 units of propylene glycol. In some embodiments, the polymer of propylene glycol comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 units of propylene glycol. In some embodiments, the polymer comprises a mixture of one or more of the polymers described herein.

In some embodiments, the polymer comprises C₆-C₁₄ isocyanate and a polymer of propylene glycol. In some embodiments, the C₆-C₁₄ isocyanate is saturated methylene diphenyldiisocyanate (SMDI) and the polymer of propylene glycol comprises 12 units of propylene glycol. In some embodiments, the polymer comprises 1 to 10 monomeric units, wherein each monomeric unit comprises SMDI and 12 units of propylene glycol. In some embodiments, the polymer comprises 1 to 8 monomeric units, wherein each monomeric unit comprises SMDI and 12 units of propylene glycol. In some embodiments, the polymer comprises 1 to 6 monomeric units, wherein each monomeric unit comprises SMDI and 12 units of propylene glycol. In some embodiments, the polymer comprises 1 to 4 monomeric units, wherein each monomeric unit comprises SMDI and 12 units of propylene glycol. In some embodiments, the polymer comprises 1, 2, 3, 4, 5, 6, 7, 8, 9, or 10 monomeric units, wherein each monomeric unit comprises SMDI and 12 units of propylene glycol.

In some embodiments, the polymer comprising polyurethane is PPG-12/SMDI, PPG-51/SMDI, HDI/trimethylol hexyllactone crosspolymer, bis-C₁₆-C₂₀ isoalkoxy TMHDI/PEG-90 polymer, bis-hydroxyethyl acrylate poly(1,4-butanediol)-9/THMDI polymer, bis-isosteryl 1,4-butanediol/HDI/hydrogenated dimer dilinoleyl alcohol polymer, bis-lauryl cocaminopropylamine/HDI/PEG-100 polymer, bis-methoxy PEG-10 dimethyl MEA/HDI/bis-PEG-10 dimethicone polymer, 1,4-butanediol/succinic acid/adipic acid/HDI polymer, cholesterol/HDI/pullulan polymer, decyl HDI/PEG-180 crosspolymer, diethylene glycol/DMAP acrylamide/PEG-180/HDI polymer, HDI/di-C₁₂-C₁₄ alkyl tartrate/hydrogenated dilinoleyl alcohol polymer, HDI/PEI-45/SMDI crosspolymer, HDI/PPG/polycaprolactone crosspolymer, methoxy PEG-17/methoxy PEG-11/HDI crosspolymer, methoxy PEG-17/methoxy PEG-11/HDI isocyanurate trimer crosspolymer, PEG-240/HDI polymer bis-decyltetradeceth-20 ether, PPG-26/HDI polymer, steareth-100/PEG-136/HDI polymer, or stearyl HDI/PEG-50 polymer. Further polymers comprising polyurethane are provided in, e.g., “Safety Assessment of Polyurethanes as Used in Cosmetics,” Draft Report for Panel Review dated Mar. 17, 2017, Cosmetic Ingredient Review; and “Safety Assessment of Hexamethylene Diisocyanate (HDI) Polymers as Used in Cosmetics,” Revised Draft Report for Panel Review dated May 13, 2016, Cosmetic Ingredient Review.

In some embodiments, the polymer is about 0.01% wt/wt to about 10% wt/wt of the formulation. In some embodiments, the polymer is about 0.02% wt/wt to about 5% wt/wt of the formulation. In some embodiments, the polymer is about 0.1% wt/wt to about 4% wt/wt of the formulation. In some embodiments, the polymer is about 0.2% wt/wt to about 3% wt/wt of the formulation. In some embodiments, the polymer is about 0.5% wt/wt to about 2% wt/wt of the formulation. In some embodiments, the polymer is about 1% wt/wt to about 2% wt/wt of the formulation. In some embodiments, the polymer is about 0.01% w/w, about 0.02% w/w, about 0.03% w/w, about 0.04% w/w, about 0.05% w/w, about 0.06% w/w, about 0.07% w/w, about 0.08% w/w, about 0.09% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.5% w/w, about 3.0% w/w, about 3.5% w/w, about 4.0% w/w, about 4.5% w/w, or about 5.0% w/w of the formulation.

In some embodiments, the polymer is capable of inhibiting skin delivery of one or more active pharmaceutical ingredients. In some embodiments, the polymer selectively inhibits the skin delivery of an active pharmaceutical ingredient while substantially unaffecting the skin delivery of a different active pharmaceutical ingredient in the same formulation. The polymer of the present formulation provides an unexpected effect of selectively inhibiting the skin delivery, and therefore minimizing systemic absorption, of desoximetasone in the formulation, while skin delivery of the tazarotene in the formulation is substantially unaffected. In some embodiments, the polymer advantageously enables co-administration of desoximetasone and tazarotene in the same formulation. In some embodiments, the polymer advantageously enables co-administration of desoximetasone and tazarotene in the same formulation, whereby the adverse side effects of dezoximetasone as described herein are reduced.

In some embodiments, the formulation comprises a solvent. In some embodiments, the solvent is an organic solvent. In some embodiments, the solvent is an organic, polyol solvent. As used herein, “organic, polyol solvent” refers to a carbon-based substance, containing multiple (e.g., more than two) hydroxyl groups, that can be used to dissolve one or more solutes. Exemplary organic, polyol solvents include, but are not limited to: diols such as, e.g., glycol, ethylene glycol, propylene glycol, isoprene glycol, butylene glycol, pentylene glycol, hexylene glycol, and caprylyl glycol; triols such as, e.g., glycerol; sugar alcohols such as, e.g., maltitol, sorbitol, xylitol, erythritol, and isomalt; and cyclitols such as, e.g., cyclopentanepentol and cyclohexanetetrol. In some embodiments, the organic, polyol solvent is selected from glycol, ethylene glycol, propylene glycol, isoprene glycol, butylene glycol, pentylene glycol, hexylene glycol, caprylyl glycol, and combinations thereof. In some embodiments, the organic, polyol solvent is hexylene glycol. In some embodiments, the solvent is any solvent except for transcutol. In some embodiments, the formulations described herein do not comprise transcutol.

In some embodiments, the solvent is about 0.1% wt/wt to about 10% wt/wt of the formulation. In some embodiments, the solvent is about 0.2% wt/wt to about 10% wt/wt of the formulation. In some embodiments, the solvent is about 0.5% wt/wt to about 10% wt/wt of the formulation. In some embodiments, the solvent is about 1% wt/wt to about 8% wt/wt of the formulation. In some embodiments, the solvent is about 2% wt/wt to about 6% wt/wt of the formulation. In some embodiments, the solvent is about 1% wt/wt, about 2% wt/wt, about 3% wt/wt, about 4% wt/wt, about 5% wt/wt, about 6% wt/wt, about 7% wt/wt, about 8% wt/wt, about 9% wt/wt, or about 10% wt/wt of the formulation.

In some embodiments, the solvent is an organic solvent. In some embodiments, the organic solvent is about 0.1% wt/wt to about 10% wt/wt of the formulation. In some embodiments, the organic solvent is about 0.2% wt/wt to about 10% wt/wt of the formulation. In some embodiments, the organic solvent is about 0.5% wt/wt to about 10% wt/wt of the formulation. In some embodiments, the organic solvent is about 1% wt/wt to about 8% wt/wt of the formulation. In some embodiments, the organic solvent is about 2% wt/wt to about 6% wt/wt of the formulation. In some embodiments, the organic solvent is about 1% wt/wt, about 2% wt/wt, about 3% wt/wt, about 4% wt/wt, about 5% wt/wt, about 6% wt/wt, about 7% wt/wt, about 8% wt/wt, about 9% wt/wt, or about 10% wt/wt of the formulation.

In some embodiments, the solvent is an organic, polyol solvent. In some embodiments, the organic, polyol solvent is about 0.1% wt/wt to about 10% wt/wt of the formulation. In some embodiments, the organic, polyol solvent is about 0.2% wt/wt to about 10% wt/wt of the formulation. In some embodiments, the organic, polyol solvent is about 0.5% wt/wt to about 10% wt/wt of the formulation. In some embodiments, the organic, polyol solvent is about 1% wt/wt to about 8% wt/wt of the formulation. In some embodiments, the organic, polyol solvent is about 2% wt/wt to about 6% wt/wt of the formulation. In some embodiments, the organic, polyol solvent is about 1% wt/wt, about 2% wt/wt, about 3% wt/wt, about 4% wt/wt, about 5% wt/wt, about 6% wt/wt, about 7% wt/wt, about 8% wt/wt, about 9% wt/wt, or about 10% wt/wt of the formulation.

In some embodiments, the desoximetasone is soluble in the solvent. In some embodiments, the tazarotene is soluble in the solvent. In some embodiments, the desoximetasone and the tazarotene are both soluble in the solvent. In some embodiments, the desoximetasone is soluble in the organic, polyol solvent. In some embodiments, the tazarotene is soluble in the organic, polyol solvent. In some embodiments, the desoximetasone and the tazarotene are both soluble in the organic, polyol solvent. As used herein, the term “soluble” refers to the ability of a substance (i.e., a solute) to dissolve in a certain solvent. One of ordinary skill in the art will appreciate that the term “soluble,” as used in reference to one or more active pharmaceutical ingredients described herein, encompasses a range of solubility for a given solute and solvent combination, for example, a solute can be considered “soluble” in a solvent when about 1000 to about 10000, or about 100 to about 1000, or about 30 to about 100, or about 10 to about 30, or about 1 to about 10, or less than about 1 parts of solvent is required to dissolve 1 part of solute.

In some embodiments, the corticosteroid is soluble in the solvent. In some embodiments, the corticosteroid and the tazarotene are both soluble in the solvent. In some embodiments, the corticosteroid is soluble in the organic, polyol solvent. In some embodiments, the cortisoteroid and the tazarotene are both soluble in the organic, polyol solvent

In some embodiments, desoximetasone has a solubility of greater than about 0.1 g/L in the solvent. In some embodiments, desoximetasone has a solubility of greater than about 1 g/L in the solvent. In some embodiments, desoximetasone has a solubility of greater than about 10 g/L in the solvent. In some embodiments, tazarotene has a solubility of greater than about 0.1 g/L in the solvent. In some embodiments, tazarotene has a solubility of greater than about 1 g/L in the solvent. In some embodiments, tazarotene has a solubility of greater than about 10 g/L in the solvent. In some embodiments, corticosteroid has a solubility of greater than about 0.1 g/L in the solvent. In some embodiments, corticosteroid has a solubility of greater than about 1 g/L in the solvent. In some embodiments, corticosteroid has a solubility of greater than about 10 g/L in the solvent

In some embodiments, desoximetasone has a solubility of greater than about 0.1 g/L in the organic, polyol solvent. In some embodiments, desoximetasone has a solubility of greater than about 1 g/L in the organic, polyol solvent. In some embodiments, desoximetasone has a solubility of greater than about 10 g/L in the organic, polyol solvent. In some embodiments, tazarotene has a solubility of greater than about 0.1 g/L in the organic, polyol solvent. In some embodiments, tazarotene has a solubility of greater than about 1 g/L in the organic, polyol solvent. In some embodiments, tazarotene has a solubility of greater than about 10 g/L in the organic, polyol solvent. In some embodiments, corticosteroid has a solubility of greater than about 0.1 g/L in the organic, polyol solvent. In some embodiments, corticosteroid has a solubility of greater than about 1 g/L in the organic, polyol solvent. In some embodiments, corticosteroid has a solubility of greater than about 10 g/L in the organic, polyol solvent.

In some embodiments, the formulation comprises about 0.01% wt/wt to about 1% wt/wt desoximetasone. In some embodiments, the formulation comprises about 0.02% wt/wt to about 0.75% wt/wt desoximetasone. In some embodiments, the formulation comprises about 0.02% wt/wt to about 0.5% wt/wt desoximetasone. In some embodiments, the formulation comprises about 0.03% wt/wt to about 0.15% wt/wt desoximetasone. In some embodiments, the formulation comprises about 0.05% wt/wt to about 0.25% wt/wt desoximetasone. In some embodiments, the formulation comprises about 0.075% wt/wt to about 0.25% wt/wt desoximetasone. In some embodiments, the formulation comprises about 0.01% wt/wt, about 0.025% wt/wt, about 0.05% wt/wt, about 0.075% wt/wt, about 0.1% wt/wt, about 0.15% wt/wt, about 0.2% wt/wt, about 0.25% wt/wt, about 0.3% wt/wt, about 0.35% wt/wt, about 0.4% wt/wt, about 0.45% wt/wt, about 0.5% wt/wt, about 0.55% wt/wt, about 0.6% wt/wt, about 0.65% wt/wt, about 0.7% wt/wt, about 0.75% wt/wt, about 0.8% wt/wt, about 0.85% wt/wt, about 0.9% wt/wt, about 0.95% wt/wt, about 1% wt/wt desoximetasone.

In some embodiments, the formulation comprises about 0.01% wt/wt to about 1% wt/wt corticosteroid. In some embodiments, the formulation comprises about 0.02% wt/wt to about 0.75% wt/wt corticosteroid. In some embodiments, the formulation comprises about 0.02% wt/wt to about 0.5% wt/wt corticosteroid. In some embodiments, the formulation comprises about 0.03% wt/wt to about 0.15% wt/wt corticosteroid. In some embodiments, the formulation comprises about 0.05% wt/wt to about 0.25% wt/wt corticosteroid. In some embodiments, the formulation comprises about 0.075% wt/wt to about 0.25% wt/wt corticosteroid. In some embodiments, the formulation comprises about 0.01% wt/wt, about 0.025% wt/wt, about 0.05% wt/wt, about 0.075% wt/wt, about 0.1% wt/wt, about 0.15% wt/wt, about 0.2% wt/wt, about 0.25% wt/wt, about 0.3% wt/wt, about 0.35% wt/wt, about 0.4% wt/wt, about 0.45% wt/wt, about 0.5% wt/wt, about 0.55% wt/wt, about 0.6% wt/wt, about 0.65% wt/wt, about 0.7% wt/wt, about 0.75% wt/wt, about 0.8% wt/wt, about 0.85% wt/wt, about 0.9% wt/wt, about 0.95% wt/wt, about 1% wt/wt corticosteroid.

In some embodiments, the formulation comprises about 0.01% wt/wt to about 1% wt/wt tazarotene. In some embodiments, the formulation comprises about 0.02% wt/wt to about 0.75% wt/wt tazarotene. In some embodiments, the formulation comprises about 0.03% % wt/wt to about 0.5% wt/wt tazarotene. In some embodiments, the formulation comprises about 0.04% wt/wt to about 0.25% wt/wt tazarotene. In some embodiments, the formulation comprises about 0.05% wt/wt to about 0.1% wt/wt tazarotene. In some embodiments, the formulation comprises about 0.01% wt/wt, about 0.025% wt/wt, about 0.05% wt/wt, about 0.075% wt/wt, about 0.1% wt/wt, about 0.15% wt/wt, about 0.2% wt/wt, about 0.25% wt/wt, about 0.3% wt/wt, about 0.35% wt/wt, about 0.4% wt/wt, about 0.45% wt/wt, about 0.5% wt/wt, about 0.55% wt/wt, about 0.6% wt/wt, about 0.65% wt/wt, about 0.7% wt/wt, about 0.75% wt/wt, about 0.8% wt/wt, about 0.85% wt/wt, about 0.9% wt/wt, about 0.95% wt/wt, about 1% wt/wt tazarotene.

In some embodiments, the formulation comprises about 0.01% wt/wt to about 1% wt/wt desoximetasone and about 0.01% wt/wt to about 1% wt/wt tazarotene. In some embodiments, the formulation comprises about 0.02% wt/wt to about 0.5% wt/wt desoximetasone and about 0.02% wt/wt to about 0.75% wt/wt tazarotene. In some embodiments, the formulation comprises about 0.03% wt/wt to about 0.15% wt/wt desoximetasone and about 0.05% wt/wt to about 0.1% wt/wt tazarotene. In some embodiments, the formulation comprises about 0.03% wt/wt desoximetasone and about 0.075% wt/wt tazarotene. In some embodiments, the formulation comprises about 0.06% wt/wt desoximetasone and about 0.075% wt/wt tazarotene. In some embodiments, the formulation comprises about 0.075% wt/wt desoximetasone and about 0.1% wt/wt tazarotene. In some embodiments, the formulation comprises about 0.15% wt/wt desoximetasone and about 0.1% wt/wt tazarotene.

In some embodiments, the formulation comprises about 0.01% wt/wt to about 1% wt/wt corticosteroid and about 0.01% wt/wt to about 1% wt/wt tazarotene. In some embodiments, the formulation comprises about 0.02% wt/wt to about 0.5% wt/wt corticosteroid and about 0.02% wt/wt to about 0.75% wt/wt tazarotene. In some embodiments, the formulation comprises about 0.03% wt/wt to about 0.15% wt/wt corticosteroid and about 0.05% wt/wt to about 0.1% wt/wt tazarotene. In some embodiments, the formulation comprises about 0.03% wt/wt corticosteroid and about 0.075% wt/wt tazarotene. In some embodiments, the formulation comprises about 0.06% wt/wt corticosteroid and about 0.075% wt/wt tazarotene. In some embodiments, the formulation comprises about 0.075% wt/wt corticosteroid and about 0.1% wt/wt tazarotene. In some embodiments, the formulation comprises about 0.15% wt/wt corticosteroid and about 0.1% wt/wt tazarotene.

In some embodiments, the formulations described herein can include any topical formulation, e.g., a viscous topical formulation. Exemplary formulations include, but are not limited to, a cream, an ointment, a gel, a lotion or a paste. In some embodiments, the formulation is an oil-in-water emulsion. In some embodiments, the formulation is a water-in-oil emulation. In some embodiments, the formulation does not comprise greater than 1%, 5% or 10% water.

In some embodiments, the topical pharmaceutical formulations provided herein further comprise a pharmaceutically acceptable excipient. A “pharmaceutically acceptable excipient,” as used herein, refers to an excipient that is compatible with the other ingredients of the formulation and does not cause significant adverse toxicological effect to the patient. For example, an excipient can be included in the formulation to achieve a desired skin delivery, e.g., penetration and permeation, stability, shelf life, metabolism, solubility, and/or release rate. Non-limiting examples of pharmaceutically acceptable excipients include buffers, co-solvents, adsorbents, penetration and/or permeation enhancers, surfactants, stabilizers, emulsifiers, preservatives, chelating agents, rheology modifiers and thickening agents, smoothening agents, ointment bases, and humectants. In some embodiments, the formulation further comprises an ointment base, a surfactant, a rheology modifier, a penetration enhancer, or combinations thereof.

In some embodiments, the formulation comprises an ointment base. In general, ointment bases are included in a formulation for their emollient, protective, and occlusive properties. Non-limiting examples of ointment bases include oleaginous ointment bases such as, e.g., white petrolatum and white ointment; absorption ointment bases such as, e.g., hydrophilic petrolatum, anhydrous lanolin, AQUABASE, AQUAPHOR, and POLYSORB; water/oil emulsion ointment bases such as, e.g., cold cream, hydrous lanolin, rose water ointment, HYDROCREAM, EUCERIN, and NIVEA; oil/water emulsion ointment bases such as, e.g., hydrophilic ointment, DERMABASE, VELVACHOL, and UNIBASE; and water-miscible ointment bases such as, e.g., PEG ointment and POLYBASE. Further examples of ointment bases are provided in, e.g., A Practical Guide to Contemporary Pharmacy Practice, 3^{rd} edition; ed. J. E. Thompson, Ch. 23: pp. 277-290; Lippincott Williams & Wilkins, January 2009. In some embodiments, the ointment base is an oleaginous ointment base having occlusive properties. In some embodiments, the ointment base is petrolatum. In some embodiments, the present disclosure provides for administration of desoximetasone in a formulation comprising an occlusive ointment base, e.g., petrolatum. In some embodiments, the present disclosure provides for administration of desoximetasone in a formulation comprising an occlusive ointment base, e.g., petrolatum, wherein the occlusive ointment base is greater than 60% of the formulation.

In some embodiments, desoximetasone is substantially soluble in the ointment base. In some embodiments, tazarotene is substantially soluble in the ointment base. In some embodiments, desoximetasone and tazarotene are both substantially soluble in the ointment base. In some embodiments, desoximetasone has a solubility of greater than about 0.1 g/L in the ointment base. In some embodiments, desoximetasone has a solubility of greater than about 1 g/L in the ointment base. In some embodiments, desoximetasone has a solubility of greater than about 10 g/L in the ointment base. In some embodiments, tazarotene has a solubility of greater than about 0.1 g/L in the ointment base. In some embodiments, tazarotene has a solubility of greater than about 1 g/L in the ointment base. In some embodiments, tazarotene has a solubility of greater than about 10 g/L in the ointment base.

In some embodiments, the corticosteroid is substantially soluble in the ointment base. In some embodiments, the corticosteroid and tazarotene are both substantially soluble in the ointment base. In some embodiments, corticosteroid has a solubility of greater than about 0.1 g/L in the ointment base. In some embodiments, corticosteroid has a solubility of greater than about 1 g/L in the ointment base. In some embodiments, corticosteroid has a solubility of greater than about 10 g/L in the ointment base.

In some embodiments, the ointment base is greater than about 50% wt/wt, greater than about 60%, greater than about 70%, or greater than about 80% of the formulation. In some embodiments, the ointment is about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% w/wt of the formulation. It was unexpectedly discovered that including an ointment base, e.g., having occlusive properties, as a major component (e.g., greater than about 50% wt/wt) in the present formulation did not substantially increase the systemic absorption of desoximetasone. The present formulation therefore has the advantageous properties of an ointment base (e.g., acting as an emollient, protective, and occlusive agent for the skin) while minimizing adverse effects due to systemic absorption of desoximetasone.

In some embodiments, the formulation comprises a surfactant. Surfactants can be included in a formulation, e.g., to improve stability of the active pharmaceutical ingredient and/or shelf life of the formulation. Non-limiting examples of surfactants include propylene glycol stearate, glyceryl monohydroxystearate, isosteareth-2, trideceth-2, trideceth-3, hydroxystearic acid, PEG-2 stearate, sorbitan monostearate, glyceryl laurate, laureth-2, cocamide monoethanolamine, lauramide monoethanolamine, sodium lauryl sulfate, polyoxyethylene stearyl ether, polysorbate, tyloxapol, poloxamer, polyoxyethylene castor oil, polyoxyl stearate, lecitin, phospholipid, sorbitan ester, and polyethoxylated fatty acid. In some embodiments, the surfactant of the formulation is selected from propylene glycol stearate, glyceryl monohydroxystearate, isosteareth-2, trideceth-2, trideceth-3, hydroxystearic acid, PEG-2 stearate, sorbitan monostearate, glyceryl laurate, laureth-2, cocamide monoethanolamine, lauramide monoethanolamine, and combinations thereof. In some embodiments, the surfactant is propylene glycol stearate.

In some embodiments, the surfactant is about 1% wt/wt to about 20% wt/wt of the formulation. In some embodiments, the surfactant is about 2% wt/wt to about 10% wt/wt of the formulation. In some embodiments, the surfactant is about 3% wt/wt to about 8% wt/wt of the formulation. In some embodiments, the surfactant is about 4% wt/wt to about 6% wt/wt of the formulation. In some embodiments, the surfactant is about 1% wt/wt, about 2% wt/wt, about 3% wt/wt, about 4% wt/wt, about 5% wt/wt, about 6% wt/wt, about 7% wt/wt, about 8% wt/wt, about 9% wt/wt, or about 10% wt/wt of the formulation.

In some embodiments, the formulation comprises a rheology modifier. Rheology modifiers, also known sometimes as thickeners or thickening agents, are typically included in a topical formulation to adjust the stability and flow properties of the formulation, and can also affect emulsion stability, water retention, water absorption, and suspension stability. In some embodiments, the rheology modifier is selected from an acrylate crosspolymer; carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC), cyclomethicone, dimethicone, dicapryl maleate, caprylic/capric triglyceride, isopropyl myristate, octyl stearate, isostearyl linoleate, medium chain triglycerides (lanolin oil, coconut oil, cocoa butter, olive oil, avocado oil, aloe extracts, jojoba oil, castor oil), fatty acid, oleic acid, stearic acid, fatty alcohol, cetyl alcohol, hexadecyl alcohol, diisopropyl adipate, hydroxybenzoate esters, benzoic acid esters, isononyl iso-nonanoate, alkanes, mineral oil, silicone, dimethyl polysiloxane, ether, polyoxypropylene butyl ether, polyoxypropylene cetyl ether, and combinations thereof. In some embodiments, the rheology modifier is castor oil. In some embodiments, the rheology modifier is coconut oil. In some embodiments, the rheology modifier is castor oil and coconut oil.

In some embodiments, the rheology modifier is about 1% wt/wt to about 20% wt/wt of the formulation. In some embodiments, the rheology modifier is about 2% wt/wt to about 15% wt/wt of the formulation. In some embodiments, the rheology modifier is about 3% wt/wt to about 10% wt/wt of the formulation. In some embodiments, the rheology modifier is about 4% wt/wt to about 9% wt/wt of the formulation. In some embodiments, the rheology modifier is about 5% wt/wt to about 8% wt/wt of the formulation. In some embodiments, the rheology modifier is about 1% wt/wt, about 2% wt/wt, about 3% wt/wt, about 4% wt/wt, about 5% wt/wt, about 6% wt/wt, about 7% wt/wt, about 8% wt/wt, about 9% wt/wt, about 10% wt/wt, about 11% wt/wt, about 12% wt/wt, about 13% wt/wt, about 14% wt/wt, about 15% wt/wt, about 16% wt/wt, about 17% wt/wt, about 18% wt/wt, about 19% wt/wt, or about 20% wt/wt of the formulation.

In some embodiments, the formulation comprises a penetration enhancer. Penetration enhancers may improve delivery of an active pharmaceutical ingredient in the formulation. Non-limiting examples of penetration enhancers include isopropyl myristate, decyl oleate, oleyl alcohol, octyldodecanol, propylene glycol, triacetin, cocoyl caprylocaprate, propylene glycol diesters of caprylic and capric acid (e.g., MIGLYOL), and diethylene glycol monoethyl ether (e.g., TRANSCUTOL). In some embodiments, the penetration enhancer is selected from propylene glycol diesters of caprylic and capric acid, diethylene glycol monoethyl ether, or combinations thereof. In some embodiments, the penetration enhancer is diethylene glycol monoethyl ether.

In some embodiments, the penetration enhancer is about 0.1% wt/wt to about 5% wt/wt of the formulation. In some embodiments, the penetration enhancer is about 0.5% wt/wt to about 4% wt/wt of the formulation. In some embodiments, the penetration enhancer is about 1% wt/wt to about 3% wt/wt of the formulation. In some embodiments, the penetration enhancer is about 0.1% wt/wt, about 0.2% wt/wt, about 0.3% wt/wt, about 0.4% wt/wt, about 0.5% wt/wt, about 0.6% wt/wt, about 0.7% wt/wt, about 0.8% wt/wt, about 0.9% wt/wt, about 1% wt/wt, about 2% wt/wt, about 3% wt/wt, about 4% wt/wt, or about 5% wt/wt of the formulation. In some embodiments, the formulation does not comprise a penetration enhancer.

In some embodiments, the present disclosure provides a topical pharmaceutical formulation comprising: (a) about 0.01% wt/wt to about 1% wt/wt desoximetasone; (b) about 0.01% wt/wt to about 1% wt/wt tazarotene; (c) about 0.02% wt/wt to about 5% wt/wt polymer comprising C₆-C₁₄ isocyanate and polypropylene glycol; and (d) about 0.2% wt/wt to about 10% wt/wt organic, polyol solvent. In some embodiments, the formulation further comprises about 60% wt/wt to about 95% wt/wt ointment base; about 2% wt/wt to about 10% wt/wt surfactant; and about 2% wt/wt to about 15% wt/wt rheology modifier. Polymers, organic, polyol solvents, ointment bases, surfactants, and rheology modifiers are further described herein.

In some embodiments, the present disclosure provides a topical pharmaceutical formulation comprising: (a) about 0.01% wt/wt to about 1% wt/wt corticosteroid; (b) about 0.01% wt/wt to about 1% wt/wt tazarotene; (c) about 0.02% wt/wt to about 5% wt/wt polymer comprising C₆-C₁₄ isocyanate and polypropylene glycol; and (d) about 0.2% wt/wt to about 10% wt/wt organic, polyol solvent. In some embodiments, the formulation further comprises about 60% wt/wt to about 95% wt/wt ointment base; about 2% wt/wt to about 10% wt/wt surfactant; and about 2% wt/wt to about 15% wt/wt rheology modifier. Polymers, organic, polyol solvents, ointment bases, surfactants, and rheology modifiers are further described herein.

In some embodiments, the present disclosure provides a method of co-administering desoximetasone and tazarotene, the method comprising topically administering a formulation comprising desoximetasone; tazarotene; a polymer comprising polyurethane; and a solvent, wherein the desoximetasone skin permeation is inhibited at least 20% compared to a formulation not comprising the polymer. Formulations comprising desoximetasone, tazarotene, a polymer comprising polyurethane, and a solvent are described herein. In some embodiments, the formulation not comprising the polymer comprises substantially the same components as the formulation comprising the polymer with the exception of the polymer. In some embodiments, the formulation not comprising the polymer comprises desoximetasone; tazarotene; and a solvent.

In some embodiments, the present disclosure provides a method of co-administering a corticosteroid and tazarotene, the method comprising topically administering a formulation comprising a corticosteroid; tazarotene; a polymer comprising polyurethane; and a solvent, wherein the corticosteroid skin permeation is inhibited at least 20% compared to a formulation not comprising the polymer. Formulations comprising corticosteroid, tazarotene, a polymer comprising polyurethane, and a solvent are described herein. In some embodiments, the formulation not comprising the polymer comprises substantially the same components as the formulation comprising the polymer with the exception of the polymer. In some embodiments, the formulation not comprising the polymer comprises corticosteroid; tazarotene; and a solvent.

In some embodiments, skin permeation of desoximetasone from the formulation comprising desoximetasone, tazarotene, a polymer comprising polyurethane, and a solvent is inhibited at least 10%, at least 20%, at least 30%, at least 40%, or at least 50% compared to a formulation not comprising the polymer. In some embodiments, skin permeation of corticosteroid from the formulation comprising corticosteroid, tazarotene, a polymer comprising polyurethane, and a solvent is inhibited at least 10%, at least 20%, at least 30%, at least 40%, or at least 50% compared to a formulation not comprising the polymer. Skin permeation of an active pharmaceutical ingredient can be measured using in vivo or in vitro tests. Suitable in vitro tests include a diffusion cell test, e.g., using static or flow-through cells; the Skin Parallel Artificial Membrane Permeability Assay (skin-PAMPA); tape stripping; microscopic methods such as two-photon scanning fluorescence microscopy, and confocal laser scanning microscopy and confocal Raman microscopy. Methods of evaluating skin penetration are further described in, e.g., Zsikó et al., Sci Pharm 87:19 (2019) and Abd et al., Clin Pharmacol 8:163-175 (2016). In some embodiments, desoximetasone skin permeation is measured by tape stripping.

Applicant has found the inhibition of desoximetasone skin penetration and permeation observed in formulations comprising a polymer comprising polyurethane was not seen with tazarotene. The polymer comprising polyurethane did not affect the tazarotene skin delivery. This observation allows for coadministration of both desoximetasone and tazarotene in a single formulation, e.g., a single gel, cream, or ointment. In some embodiments, skin delivery of tazarotene in the formulation comprising desoximetasone, tazarotene, a polymer comprising polyurethane, and a solvent is not substantially inhibited compared to a formulation not comprising the polymer. A person of ordinary skill in the art will understand that in the context of skin delivery, “not substantially inhibited” means that an approximately equivalent amount or a bioequivalent amount, of an active pharmaceutical ingredient, e.g., tazarotene, permeates and/or penetrates the skin using the skin permeation methods described herein. In some embodiments, the polymer advantageously enables co-administration of desoximetasone and tazarotene in the same formulation.

In some embodiments, skin permeation and/or penetration of desoximetasone in the formulation comprising desoximetasone, tazarotene, a polymer comprising polyurethane, and a solvent is inhibited compared to a formulation not comprising the polymer or tazarotene. In some embodiments, skin permeation and/or penetration of a corticosteroid in the formulation comprising corticosteroid, tazarotene, a polymer comprising polyurethane, and a solvent is inhibited compared to a formulation not comprising the polymer or tazarotene.

In some embodiments, the present disclosure provides a method of co-administering desoximetasone and tazarotene, the method comprising topically administering a formulation comprising desoximetasone; tazarotene; a polymer comprising polyurethane; and a solvent, wherein the hypothalamic pituitary adrenal (HPA) axis suppression is reduced at least 10% compared to a formulation not comprising the polymer. Formulations comprising desoximetasone, tazarotene, a polymer comprising polyurethane, and a solvent, as well as formulations not comprising the polymer, are described herein. In some embodiments, the present disclosure provides a method of co-administering a corticosteroid and tazarotene, the method comprising topically administering a formulation comprising corticosteroid; tazarotene; a polymer comprising polyurethane; and a solvent, wherein the hypothalamic pituitary adrenal (HPA) axis suppression is reduced at least 10% compared to a formulation not comprising the polymer.

In some embodiments, the HPA axis suppression of the formulation comprising desoximetasone, tazarotene, a polymer comprising polyurethane, and a solvent is reduced at least 10%, at least 20%, at least 30%, at least 40%, or at least 50% compared to a formulation not comprising the polymer. In some embodiments, the HPA axis suppression of the formulation comprising a corticosteroid, tazarotene, a polymer comprising polyurethane, and a solvent is reduced at least 10%, at least 20%, at least 30%, at least 40%, or at least 50% compared to a formulation not comprising the polymer. HPA axis suppression can be determined, for example, by measuring the levels of free and total serum cortisol, urinary free cortisol, salivary cortisol, adrenocorticotropic hormone (ACTH), cortisol binding globulin, 11-deoxycortisol, anti-adrenal antibodies, corticotrophin releasing hormone (CRH). Methods of determining HPA axis suppression are further described, e.g., in Nelson et al., J Clin Pharmacol 42:319-326 (2002) and Yeo et al., “Endocrine Testing Protocols: Hypothalamic Pituitary Adrenal Axis.” Copyright 2002-2019 MDText.com, Inc. In some embodiments, the reduced HPA axis suppression of the formulation comprising desoximetasone, tazarotene, a polymer comprising polyurethane, and a solvent is achieved by inhibition of the desoximetasone skin permeation and/or penetration by the polymer.

All references cited herein, including patents, patent applications, papers, textbooks and the like, and the references cited therein, to the extent that they are not already, are hereby incorporated herein by reference in their entirety.

### EXAMPLES

### Example 1. Formulations to Test the Effect of PPG-12/SMDI

Formulations comprising desoximetasone and tazarotene were prepared according to Table 1 and used in the subsequent Examples.

TABLE 1

| | % w/w | % w/w | % w/w | % w/w | % w/w | % w/w | % w/w | % w/w |
|---|---|---|---|---|---|---|---|---|
| | Formulation ID | | | | | | | |
| Ingredient | 1 | 2 | 3 | 4 | 5 | 6 | 7 | 8 |
| Tazarotene | 0.1 | 0.1 | 0.1 | 0.1 | 0.1 | 0.1 | 0.075 | 0.075 |
| (API) | | | | | | | | |
| Desoximetasone | 0.075 | 0.15 | 0.15 | 0.075 | 0.075 | 0.15 | 0.03 | 0.06 |
| (API) | | | | | | | | |
| ointment base | 80-95 | 80-95 | 80-95 | 80-95 | 80-95 | 80-95 | 80-95 | 80-95 |
| PPG-12/SMDI | 0.5 | 0.5 | 0.0 | 0.0 | 0.0 | 1.5 | 1.5 | 1.5 |
| rheology modifier | 0.5-10 | 1-10 | 1-10 | 1-10 | 1-10 | 1-10 | 1-10 | 1-10 |
| solvent | 1-10 | 1-10 | 1-10 | 1-10 | 1-10 | 1-10 | 1-10 | 1-10 |
| surfactant | 1-10 | 1-10 | 1-10 | 1-10 | 1-10 | 1-10 | 1-10 | 1-10 |
| penetration | 1-5 | 0.0 | 0.0 | 0.0 | 1-5 | 0.0 | 0.0 | 0.0 |
| enhancer | | | | | | | | |

### Example 2. Skin Permeation/Penetration—Study I, Human Skin

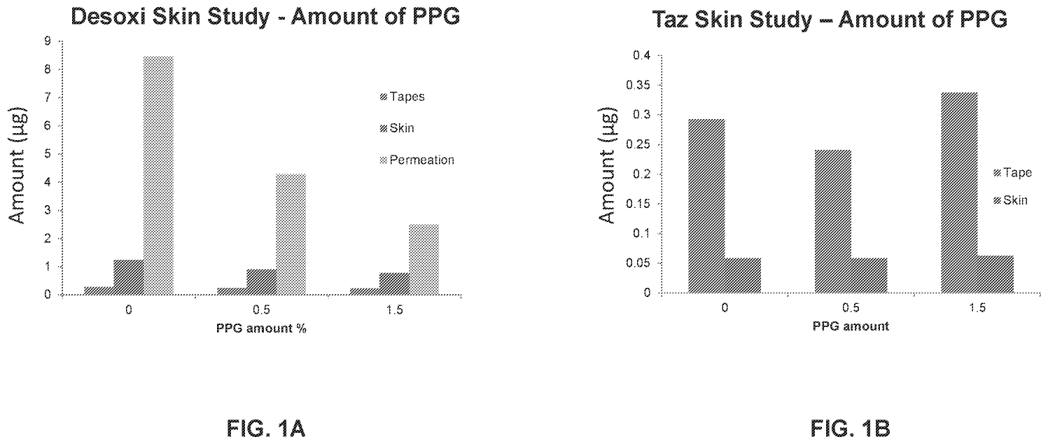

Formulations 3, 2, 6, each containing 0.15% desoximetasone, 0.1% Tazarotene, and 0%, 0.5%, and 1.5% PPG-12/SMDI, respectively, were tested for skin penetration/permeation using human skin in Franz cell setup. The mass balance results for desoximetasone are shown in FIG. 1a and summarized in Table 1. Results for Tazarotene are shown in FIG. 1b.

TABLE 1

| | (0.15% | (0.15% | (0.15% |
|---|---|---|---|
| | desoxi- | desoxi- | desoxi- |
| | metasone, | metasone, | metasone, |
| Summary/ | 0% PPG- | 0.5% PPG- | 1.5% PPG- |
| Formulation | 12/SMDI) | 12/SMDI) | 12/SMDI) |
| Number | 3 | 2 | 6 |
| Wash (μg/cm²) | 5.19 | 10.65 | 13.18 |
| Tape (μg/cm²) | 0.28 | 0.24 | 0.24 |
| Skin (μg/cm²) | 1.24 | 0.91 | 0.78 |
| Permeation | 8.46 | 4.29 | 2.49 |
| (μg/cm²) | | | |

The results indicate that increasing the amount of PPG-12/SMDI in the ointment formulations decreases the total delivery of desoximetasone only. No effect was observed with tazarotene. Therefore, unexpectedly the inhibitory effect of PPG-12/SMDI on skin penetration and/or permeation is selective to Desoximetasone, while no effect of is observed with Tazarotene.

### Example 3. Skin Permeation/Penetration—Study II, Human Skin

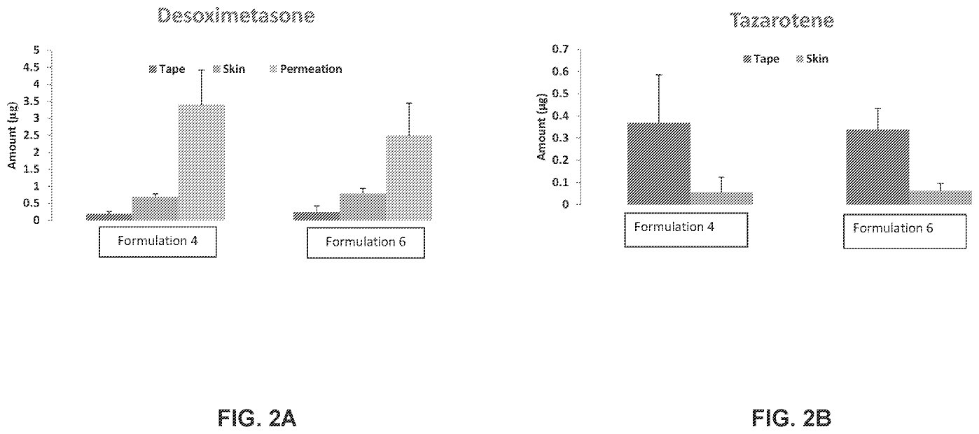

Formulations 4 and 6 in Example 1 were tested for skin permeation/penetration using human skin in Franz cell setup. Formulation 4 contains desoximetasone 0.075%/Tazarotene 0.1% without PPG-12/SMDI. Formulation 6 contains desoximetasone 0.15%/Tazarotene 0.1% with 1.5% PPG-12/SMDI. As revealed from Table 2 and FIG. 2, due to the presence of PPG-12/SMDI polymer in Formulation 6 (0.15% desoximetasone) it expressed lower permeation through the skin of Desoximetasone as compared to Formulation 4 (0.075% Desoximetasone) even though it has double the amount of Desoximetasone. No effect of PPG-12/SMDI was observed with the penetration/permeation of Tazarotene.

TABLE 2

| | Desoximetasone (μg) | | Tazarotene (μg) | |
|---|---|---|---|---|
| | 4- | | 4- | |
| | desoximetasone | 6- | desoximetasone | 6- |
| | 0.075%/ | desoximetasone | 0.075%/ | desoximetasone |
| | Tazarotene 0.1% | 0.15%/Tazarotene | Tazarotene 0.1% | 0.15%/Tazarotene |
| Formulation | without PPG- | 0.1% with 1.5% | without PPG- | 0.1% with 1.5% |
| No | 12/SMDI | PPG-12/SMDI | 12/SMDI | PPG-12/SMDI |
| Tape (μg/cm²) | 0.191 | 0.235 | 0.368 | 0.337 |
| Skin (μg/cm²) | 0.687 | 0.783 | 0.055 | 0.062 |
| Permeation | 3.398 | 2.489 | Not detected | Not detected |
| (μg/cm²) | | | | |

### Example 4. Clinical Study—HPAaxis Suppression

Formulations 6, 7, and 8, which have the same excipients (including 1.5% PPG-12/SMDI) and 0.15%, 0.03%, and 0.06% of desoximetasone, respectively, and Formulation 4, which contains 0.075% desoximetasone and no PPG-12/SMDI, were used in a clinical study. The formulations were administered once daily for 8 and 12 weeks. The subjects were tested for HPA axis suppression potential. Results are shown in Table 3.

TABLE 3

| | | | | HPA Axis |
|---|---|---|---|---|
| | % w/w | | | Suppression |
| Formu- | Desoxi- | % w/w | | (%) |
| lation | metasone | Tazarotene | Polymer | Week 8 or 12 |
| 6 | 0.15 | 0.1 | 1.5% | below 45% |
| 8 | 0.06 | 0.075 | 1.5% | below 15% |
| 7 | 0.03 | 0.075 | 1.5% | Below 10% |
| 4 | 0.075 | 0.1 | — | Above 50% |

The results indicate that HPA axis suppression by desoximetasone is dose-related for Formulations 6, 8, and 7, which have the same excipients and different amounts of desoximetasone. Although Formulation 4 contains 0.075% Desoximetasone, it exhibits the highest HPAaxis suppression, even higher than Formulation 6 which has double amount of Desoximetasone. Therefore, there is direct correlation between the specific inhibitory effect of PPG-12/SMDI on Desoximetasone skin penetration/permeation and the reduction in HPAaxis suppression in patients treated with a formulation containing PPG-12/SMDI. Since HPAaxis suppression is the result of systemic absorption of Desoximetasone, the reduction in HPAaxis suppression is advantageous from safety point of view.

All references cited herein, including patents, patent applications, papers, textbooks and the like, and the references cited therein, to the extent that they are not already, are hereby incorporated herein by reference in their entirety.

What is claimed is:

1. A topical pharmaceutical formulation comprising:
a. about 0.01% wt/wt to about 1% wt/wt desoximetasone;
b. about 0.01% wt/wt to about 1% wt/wt tazarotene;
c. a polymer comprising polyurethane; and
d. a solvent.

2. The formulation of claim 1, wherein the polymer is a bis-urethane polyol polymer.

3. The formulation of claim 1, wherein the polymer comprising polyurethane comprises a polyether.

4. The formulation of claim 1, wherein the polymer comprising polyurethane comprises polypropylene glycol.

5. The formulation of claim 1, wherein the polymer comprises C₆-C₁₄ isocyanate and a polymer of propylene glycol.

6. The formulation of any one of claims 4 or 5, wherein the polymer of propylene glycol comprises 6 to 20 units of propylene glycol.

7. The formulation of any one of claims 4 or 5, wherein the polymer of propylene glycol comprises 10 to 14 units of propylene glycol.

8. The formulation of any one of claims 4 or 5, wherein the polymer of propylene glycol comprises 12 units of propylene glycol.

9. The formulation of claim 5, wherein the C₆-C₁₄ isocyanate is selected from trimethylhexanediisocyanate (TMHDI), saturated methylene diphenyldiisocyanate (SMDI), and hexamethylene diisocyante (HDI) trimer.

10. The formulation of claim 9, wherein the C₆-C₁₄ isocyanate is saturated methylene diphenyldiisocyanate (SMDI).

11. The formulation of any one of claims 5 to 10, wherein the C₆-C₁₄ isocyanate is saturated methylene diphenyldiisocyanate (SMDI) and the polymer of propylene glycol comprises 12 units of propylene glycol.

12. The formulation of any one of claims 1 to 11, wherein the polymer is about 0.02% wt/wt to about 5% wt/wt.

13. The formulation of any one of claims 1 to 12, wherein the polymer is about 0.5% wt/wt to about 2% wt/wt.

14. The formulation of any one of claims 1 to 13, wherein the polymer is about 1% wt/wt to about 2% wt/wt.

15. The formulation of any one of claims 1 to 14, wherein the solvent is an organic solvent.

16. The formulation of any one of claims 1 to 15, wherein the solvent is an organic, polyol solvent.

17. The formulation of any one of claims 1 to 16, wherein the solvent is selected from glycol, ethylene glycol, propylene glycol, isoprene glycol, butylene glycol, pentylene glycol, hexylene glycol, caprylyl glycol, and combinations thereof.

18. The formulation of claim 17, wherein the solvent is hexylene glycol.

19. The formulation of any one of claims 1 to 18, wherein the solvent is about 0.2% wt/wt to about 10% wt/wt.

20. The formulation of any one of claims 1 to 19, wherein the solvent is about 2% wt/wt to about 6% wt/wt.

21. The formulation of any one of claims 1 to 20, wherein the desoximetasone is soluble in the solvent.

22. The formulation of any one of claims 1 to 21, wherein the tazarotene is soluble in the solvent.

23. The formulation of any one of claims 1 to 23, wherein the desoximetasone and the tazarotene are both soluble in the solvent.

24. The formulation of any one of claims 1 to 23, further comprising an ointment base, a surfactant, a rheology modifier, a penetration enhancer, or combinations thereof.

25. The formulation of claim 24, wherein the ointment base is petrolatum.

26. The formulation of claim 24, wherein the ointment base is greater than 60% wt/wt of the formulation.

27. The formulation of any one of claims 24 to 26, wherein the tazarotene is soluble in the ointment base.

28. The formulation of claim 24, wherein the surfactant is selected from propylene glycol stearate, glyceryl monohydroxystearate, isosteareth-2, trideceth-2, trideceth-3, hydroxystearic acid, PEG-2 stearate, sorbitan monostearate, glyceryl laurate, laureth-2, cocamide monoethanolamine, lauramide monoethanolamine, and combinations thereof.

29. The formulation of claim 28, wherein the surfactant is propylene glycol stearate.

30. The formulation of claim 24, wherein the rheology modifier is selected from an acrylate crosspolymer; carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC), cyclomethicone, dimethicone, dicapryl maleate, caprylic/capric triglyceride, isopropyl myristate, octyl stearate, isostearyl linoleate, medium chain triglycerides (lanolin oil, coconut oil, cocoa butter, olive oil, avocado oil, aloe extracts, jojoba oil, castor oil), fatty acid, oleic acid, stearic acid, fatty alcohol, cetyl alcohol, hexadecyl alcohol, diisopropyl adipate, hydroxybenzoate esters, benzoic acid esters, isononyl iso-nonanoate, alkanes, mineral oil, silicone, dimethyl polysiloxane, ether, polyoxypropylene butyl ether, polyoxypropylene cetyl ether, and combinations thereof.

31. The formation of claim 30, wherein the rheology modifier is castor oil.

32. The formulation of claim 30, wherein the rheology modifier is coconut oil.

33. The formulation of claim 24, wherein the penetration enhancer is selected from propylene glycol diesters of caprylic and capric acid, diethylene glycol monoethyl ether, or combinations thereof.

34. A topical pharmaceutical formulation comprising:
a. about 0.03% wt/wt to about 0.15% wt/wt desoximetasone;
b. about 0.05% wt/wt to about 0.1% wt/wt tazarotene;
c. about 0.02% wt/wt to about 5% wt/wt polymer comprising C₆-C₁₄ isocyanate and polypropylene glycol; and
d. about 0.2% wt/wt to about 10% wt/wt organic, polyol solvent.

35. The topical pharmaceutical formulation of claim 34, further comprising:
e. about 60% wt/wt to about 95% wt/wt ointment base,
f. about 2% wt/wt to about 10% wt/wt surfactant; and
g. about 2% wt/wt to about 15% wt/wt rheology modifier.

36. A method of co-administering desoximetasone and tazarotene, the method comprising topically administering a formulation comprising desoximetasone; tazarotene; a polymer comprising polyurethane; and a solvent, wherein the desoximetasone skin permeation through the skin is inhibited at least 20% compared to a formulation not comprising the polymer.

37. The method of claim 36, wherein the skin permeation of the tazarotene is not substantially inhibited compared to a formulation not comprising the polymer.

38. A method of co-administering desoximetasone and tazarotene, the method comprising topically administering a formulation comprising desoximetasone; tazarotene; a polymer comprising polyurethane; and a solvent, wherein the hypothalamic pituitary adrenal (HPA) axis suppression is reduced at least 10% compared to a formulation not comprising the polymer.
